(19) United States
(12) Patent Application Publication
(10) Pub. No.: US 2021/0402185 A1
(43) Pub. Date: Dec. 30, 2021

(54) ACTIVITY CLASSIFICATION OF BALANCE PROSTHESIS RECIPIENT

(71) Applicant: Cochlear Limited, Macquarie University, NSW (AU)

(72) Inventors: Koen Erik Van den Heuvel, Hove (BE); Joerg Pesch, Mechelen (BE)

(21) Appl. No.: 17/294,070

(22) PCT Filed: Apr. 20, 2020
(86) PCT No.: PCT/IB2020/053725

§ 371 Date: May 14, 2021

Related U.S. Application Data
(60) Provisional application No. 62/838,427, filed on Apr. 25, 2019.

(51) Int. Cl. A61N 1/36; A61N 1/378; A61N 1/05; A61B 5/11; A61B 5/00

(52) U.S. Cl. CPC A61N 1/36038; A61N 1/3787; A61N 1/36175; A61B 2562/0219; A61B 5/1118; A61B 5/7264; A61N 1/0541

(57) ABSTRACT

Presented herein are techniques for stimulating a balance prosthesis recipient based on one or more motion signals and a classification of the type of activity in which the recipient is currently participating. More specifically, a balance prosthesis system is configured to monitor the motion of at least part of a recipient's body and to determine an activity classification for the recipient (e.g., determine the “class” or “category” of the recipient's real-time motion). The recipient's motion and the activity classification are used to generate stimulation signals for delivery to the recipient.

### BACKGROUND

### Field of the Invention

The present invention generally relates to implantable balance prostheses.

### Related Art

Medical devices having one or more implantable components, generally referred to herein as implantable medical devices, have provided a wide range of therapeutic benefits to recipients over recent decades. In particular, partially or fully-implantable medical devices such as hearing prostheses (e.g., bone conduction devices, mechanical stimulators, cochlear implants, etc.), implantable pacemakers, defibrillators, functional electrical stimulation devices, and other implantable medical devices, have been successful in performing lifesaving and/or lifestyle enhancement functions and/or recipient monitoring for a number of years.

The types of implantable medical devices and the ranges of functions performed thereby have increased over the years. For example, many implantable medical devices now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, the implantable medical device.

### SUMMARY

In one aspect, a method is provided. The method comprises: capturing, with at least one motion sensor, one or more motion signals representing motion of a vestibular implant recipient; determining, based on the one or more motion signals, an activity classification of the recipient's current activity; and based on the motion signals and the activity classification, generating electrical stimulation signals for delivery to the recipient's vestibular system.

In another aspect, a vestibular stimulation system is provided. The vestibular stimulation system comprises: one or more motion sensors configured to convert motion of a recipient of the vestibular stimulation system into one or more motion signals; at least one activity classifier configured to generate, based on the one or more motion signals, an activity classification representing a real-time activity of the recipient; at least one processor configured to generate stimulation control signals based on the motion signals and the activity classification; and a stimulator unit configured to convert the stimulation control signals into electrical stimulation signals for delivery to the recipient's vestibular system.

In another aspect, a method is provided. The method comprises: monitoring motion of a head of a recipient of a vestibular implant; generating, based on the motion of the head of the recipient, a categorization of an activity being performed by the recipient; and generating vestibular stimulation signals based on the motion of the head of the recipient and categorization of the activity being performed by the recipient.

### BRIEF DESCRIPTION OF THE DRAWINGS

Embodiments of the present invention are described herein in conjunction with the accompanying drawings, in which:

FIG. 1A is a schematic diagram illustrating anatomical structures of the human vestibular system;

FIG. 1B is a schematic cross-sectional view illustrating further details of a portion of the human vestibular system of FIG. 1A;

FIG. 2A is a schematic diagram illustrating a vestibular stimulation system, in accordance with certain embodiments presented herein;

FIG. 2B is a simplified block diagram of the vestibular stimulation system of FIG. 2A, in accordance with certain embodiments presented herein;

FIG. 2C is a schematic diagram illustrating a stimulation arrangement of the vestibular stimulation system of FIG. 2A, in accordance with certain embodiments presented herein;

FIG. 3 is a functional block diagram of a portion of the vestibular stimulation system of FIG. 2A, in accordance with certain embodiments presented herein;

FIG. 4 is a functional block diagram of a vestibular stimulation system, in accordance with certain embodiments presented herein;

FIG. 5 is a functional block diagram of another vestibular stimulation system, in accordance with certain embodiments presented herein;

FIG. 6 is a functional block diagram of another vestibular stimulation system, in accordance with certain embodiments presented herein;

FIG. 7 is a flowchart of a method, in accordance with certain embodiments presented herein; and

FIG. 8 is a flowchart of a method, in accordance with certain embodiments presented herein.

### DETAILED DESCRIPTION

Presented herein are techniques for stimulating a balance prosthesis recipient based on one or more motion signals and a classification of the type of activity in which the recipient is currently participating. More specifically, a balance prosthesis system is configured to monitor the motion of at least part of a recipient's body and to determine an activity classification for the recipient (e.g., determine the “class” or “category” of the recipient's real-time motion). The recipient's motion and the activity classification are used to generate stimulation signals for delivery to the recipient.

As used herein, a “balance prosthesis” or “balance implant” is a medical device that is configured to assist recipients who suffer from balance disorders. A balance disorder is a condition in which an individual lacks the ability to control and/or maintain proper body position in a comfortable manner. Balance problems can manifest in different manners, such as feelings of unsteadiness or dizziness, a feeling of movement, spinning, or floating, even though standing still or lying down, falling, blurred vision, inability to stand or walk un-aided, etc. Balance disorders can be caused by certain health conditions, medications, aging, infections, head injuries, problems in the inner ear, problems with brain or the heart, problems with blood circulation, etc.

Different balance prosthesis are being developed to treat different types/causes of balance disorders. For example, vestibular stimulation systems are medical device systems that are used to treat balance disorders resulting from a complete or partial loss of vestibular function/sensation in one or both ears. Vestibular stimulation systems measure head movement and convert the head movement into electrical stimulation signals. The electrical stimulation signals are delivered to the recipient's vestibular system via one or more implanted electrodes. As such, the one or more electrodes stimulate the vestibular nerve, creating signals that help the brain to compensate for the loss of vestibular function.

Another type of balance prosthesis is designed to simulate the movement of fluid within the semicircular canal. In a normal ear, fluid changes help the brain understand the movement and position of the head. These balance prostheses combine microcontroller circuitry with one or more mechanical devices that function to increase normal fluid movement in the semicircular canals, thereby providing a stronger vestibular signal to the brain.

Merely for ease of description, the techniques presented herein are primarily described herein with reference to one illustrative balance prosthesis system, namely a vestibular stimulation system. However, it is to be appreciated that the techniques presented herein may also be used with a variety of other types of medical devices, including other balance prosthesis systems.

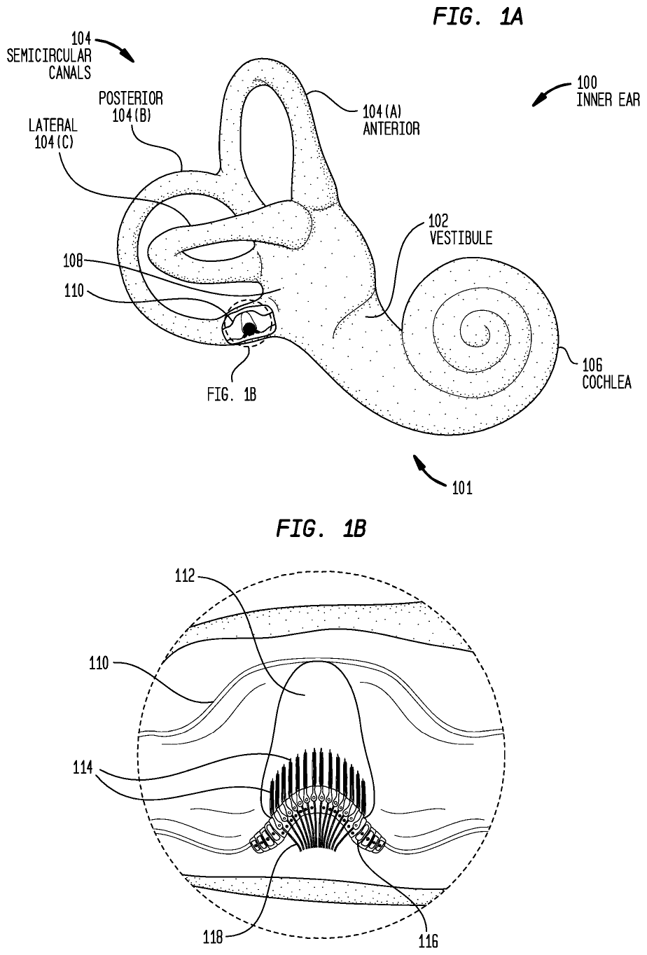

Before describing details of the techniques presented herein, relevant aspects of an example inner ear 100 in which components of a vestibular stimulation system may be implanted are first described below with reference to FIGS. 1A and 1B. More specifically, FIG. 1A is a first perspective view of the inner ear 100, while FIG. 1B is a schematic cross-sectional view illustrating further details of a portion of the vestibular system.

The bony labyrinth 101 is the rigid, bony outer wall of the inner ear 100 in the temporal bone. The bony labyrinth 101 includes three sections/parts, referred to as the vestibule 102, the semicircular canals 104, and the cochlea 106. These are cavities hollowed out of the substance of the bone, and lined by periosteum.

The semicircular canals 104 are three half-circular, interconnected tubes located adjacent cochlea 106. The three canals are the superior or anterior semicircular canal 104(A), the posterior semicircular canal 104(B), and the horizontal or lateral semicircular canal 104(C). The three canals 104(A), 104(B), and 104(C) are aligned approximately orthogonally to one another (i.e., at right angles to each other) so that they measure motions in all three planes. Specifically, lateral canal 104(C) is aligned roughly horizontally in the head, while the superior 104(A) and posterior canals 104(B) are aligned roughly at a 45 degree angle to a vertical through the center of the individual's head.

The vestibule 102 and the semicircular canals 104 are involved in the sense of equilibrium. Each of the vestibule 102 and the semicircular canals 104 has an organ containing hair cells. In particular, the utricle and saccule (i.e., two saclike structures, located in the vestibule 102) each contain a macula, an organ consisting of a patch of hair cells covered by a gelatinous membrane containing particles of calcium carbonate, called otoliths. Motions of the head cause the otoliths to pull on the hair cells, stimulating the vestibular nerve (not shown in FIG. 1A), which signals the position of the head with respect to the rest of the body.

Within each semicircular canal 104 is a semicircular duct filled with a fluid called endolymph and, upon rotation of the head with a component of motion in the appropriate direction, fluid is caused to move within the canal. At the base of each canal 104 is the ampula 108 and the related crista 110, which is shown in greater detail in FIG. 1B. Within the crista 110 is the cupula 112 which contains hair bundles 114 connected to hair cells 116, and in turn to nerve fibres 118. When the fluid moves, the hair cells 116 are stimulated, and produce a corresponding neural signal.

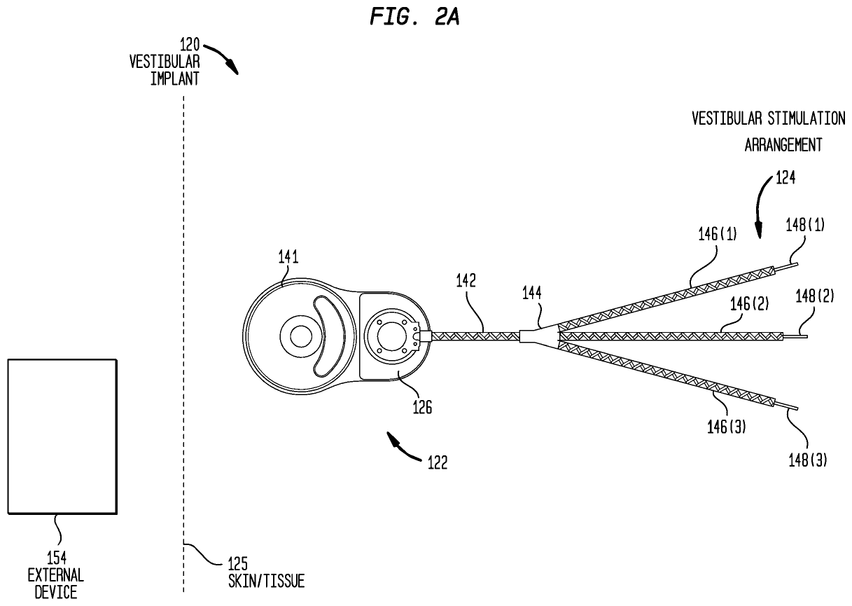

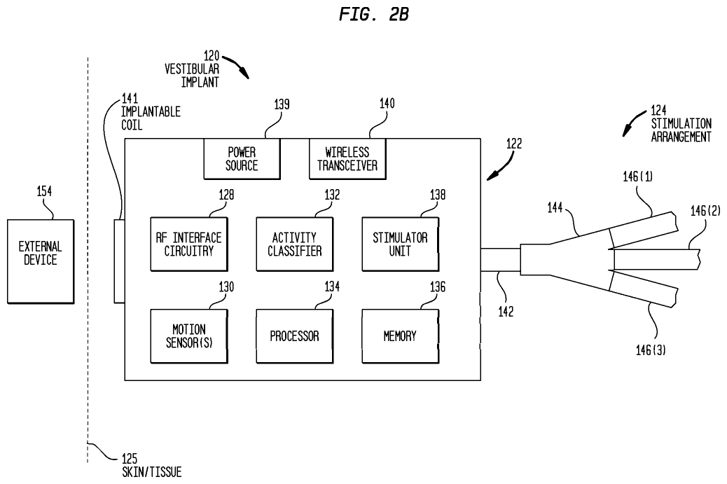

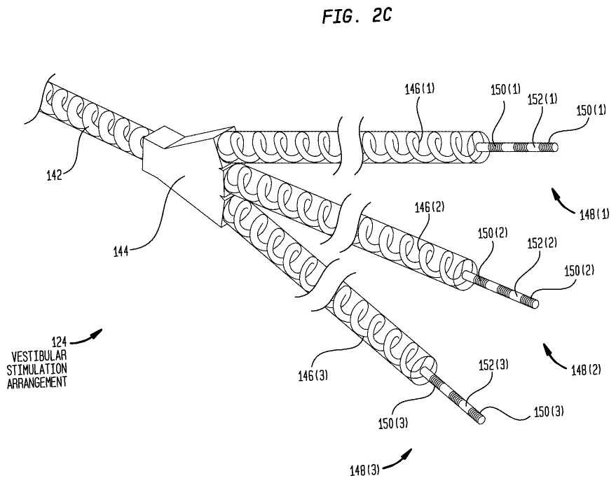

As noted, the vestibule 102 and the semicircular canals 104 sense head tilt and rotation during movement, which in turn helps the individual maintain balance, stabilize vision, etc. However, certain individuals may suffer from a balance disorder with complete or partial loss of vestibular function/sensation in one or both ears. This loss of vestibular function leads to imbalance/instability problems, dizziness, difficulty walking in darkness without falling, blurred or unsteady vision during head movement, etc. Presented herein are vestibular stimulation systems that are configured to replace or supplement vestibular function through direct stimulation (e.g., electrical stimulation) of a recipient's vestibular system. In particular, as described further below, a vestibular stimulation system in accordance with embodiments presented herein senses/measures recipient motion/movement, generates a classification/categorization of the motion, and stimulates the vestibular nerve in the inner ear with an electrode array atraumatically implanted within one or more of semicircular canals 104. The electrical stimulation is delivered in a manner that restores vestibular function (i.e., replicates the balance sensory implants provided to the brain via a fully functional vestibular system). FIGS. 2A and 2B is illustrate further details of one such example vestibular implant.

More specifically, shown in FIG. 2A is a perspective view of a vestibular stimulation system 115, which includes a vestibular implant (implantable component) 120. FIG. 2B is a block diagram of the vestibular implant 120, while FIG. 2C is a schematic diagram of a portion of the vestibular implant 120. For ease of description, FIGS. 2A, 2B, and 2C will be described together.

As shown, the vestibular implant 120 comprises an implant body (main module) 122 and a vestibular stimulation arrangement 124, both of which are implantable within a recipient (i.e., implanted under the skin/tissue 125 of a recipient). The implant body 122 generally comprises a hermetically-sealed housing 126 in which Radio-Frequency (RF) interface circuitry 128, one or more motion sensors 130, an activity classifier 132, at least one processor 134, memory 136, a stimulator unit 138, a rechargeable power source 139, and a wireless transmitter/receiver (transceiver) 140 are disposed. The implant body 122 also includes an internal/implantable coil 141 that is generally external to the housing 126, but which is connected to the RF interface circuitry 128 via a hermetic feedthrough (not shown in FIG. 2B).

Each of the activity classifier 132 and the processor 134 may be formed by one or more processors (e.g., one or more Digital Signal Processors (DSPs), one or more uC cores, etc.), firmware, software, etc. arranged to perform operations described herein. That is, the activity classifier 132 and the processor 134 may each be implemented as firmware elements, partially or fully implemented with digital logic gates in one or more application-specific integrated circuits (ASICs), partially in software, etc.

FIG. 2C is an enlarged, perspective view of the vestibular stimulation arrangement 124. As shown, the vestibular stimulation arrangement 124 comprises a primary lead 142 which trifurcates at junction 144 into three (3) secondary or electrode leads 146(1), 146(2), and 146(3). The secondary leads 146(1), 146(2), and 146(3) each terminate in an electrode assembly 148(1), 148(2), and 148(3) each configured to be inserted into one of the recipient's semicircular canals. That is, the vestibular stimulation arrangement 124 comprises a number of small electrode assemblies for surgical placement, for example, between the bony labyrinth and the membranous labyrinth of each semicircular canal (superior, posterior and lateral) of the vestibular labyrinth.

The electrode assembly 148(1) comprises a plurality of electrodes 150(1) disposed in a carrier member 152(1) (e.g., a flexible silicone body). Similarly, electrode assembly 148(2) comprises a plurality of electrodes 150(2) disposed in a carrier member 152(2), while electrode assembly 148(3) comprises a plurality of electrodes 150(3) disposed in a carrier member 152(3). In this specific example, the electrode assemblies 148(1)-148(3) each comprise three (3) electrodes, which function as an electrical interface to the vestibular periphery without damaging or destroying residual vestibular function. It is to be appreciated that this specific embodiment with three electrodes in each of the electrode assemblies 148(1)-148(3) is merely illustrative and that the techniques presented herein may be used with stimulating assemblies having different numbers of electrodes, stimulating assemblies having different lengths, etc.

In general, the electrode assemblies 148(1)-148(3) are configured such that a surgeon can implant one, two, or all three of the electrode assemblies into to either one, two or all three of the semicircular canals. The trifurcated leads 146(1)-146(3) allows for ease of surgical placement and improves lead reliability (impact, fatigue, stress, etc.).

It is desirable that the electrode assemblies 148(1)-148(3) sufficient stiffness and dynamics such that the electrode assemblies 148(1)-148(3) can be placed reliably within the semicircular canals. In certain examples, the electrode assemblies 148(1)-148(3) include stiffening members allowing the electrode assemblies 148(1)-148(3) to have sufficient stiffness to insert to the desired depth between the bony labyrinth and the membranous labyrinth of each semicircular canal. In general, the electrode assemblies 148(1)-148(3) each have a stiffness allowing a single stroke atraumatic insertion to the required depth in the semicircular canals. However, the electrode assemblies 148(1)-148(3) also have sufficient flexibility to deflect and avoid damage to the delicate anatomical structures.

As noted above, the vestibular implant 120 comprises RF interface circuitry 128 and a rechargeable power source 139 (e.g., one or more rechargeable batteries). The power source 139 is recharged using power received from an external device 154 via the RF interface circuitry 128. That is, although not shown in FIG. 2B, the external device 154 comprises an external coil configured to be inductively coupled with the implantable coil 141. When inductively coupled, the external coil and the implantable coil 141 form a closely-coupled wireless link by which power is transferred from a power source of the external device through the skin/tissue 125 of the recipient. In certain examples, the closely-coupled wireless link is a radio frequency (RF) link. However, various other types of energy transfer, such as infrared (IR), electromagnetic, capacitive and inductive transfer, may be used to transfer the power and/or data from the external device to the vestibular implant 120.

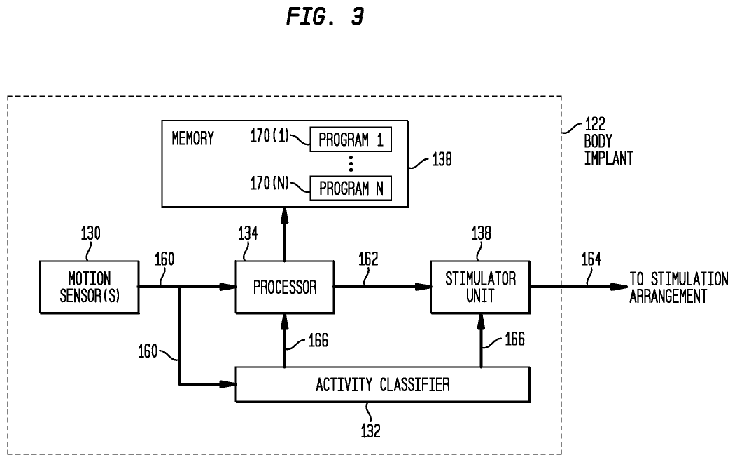

Also as noted, the vestibular implant 120 comprises one or more motion sensors 130, an activity classifier 132, a processor 134, memory 136, and stimulator unit 138. In general, these components are used by the vestibular implant 120 to electrically stimulate the vestibular system of the recipient to, for example, restore (e.g., replace or supplement) vestibular function. FIG. 3 is a functional block diagram illustrating operation of these components in accordance with certain embodiments presented herein.

More specifically, referring to FIG. 3, the one or more motion sensors 130 may include, for example, translation or velocity sensors for sensing translation of the recipient's head and/or rotation sensors for sensing rotation of the recipient's head. The one or more motion sensors 130 may comprise, for example, one or more accelerometers, one or more gyroscopes, one or more magnetometers, etc. In certain example, the one or more motion sensors 130 are configured to sense the recipient's orientation and translation/velocity along three coordinate axes and (i.e., sense the recipient's roll, pitch and yaw).

The one or more motion sensors 130 monitor the movement/motion of the recipient's head and, as such, generate one or more motion signals 160 that depend on one or both of the rotational and translational motion experienced by the recipient. That is, the one or more motion signals 160 include translation and/or rotation data representing the motion experienced by the recipient. The motion signals 160 are then provided to the processor 134.

The processor 134 is configured to analyze the one or more motion signals 160 and to perform a number of operations. In particular, the processor 134 is configured to, based on the motion signals 160 (e.g., data representing recipient's orientation, velocity, etc.), generate stimulation control signals 162 representing electrical stimulation that is to be delivered to the recipient. That is, the processor 134 execute operation instructions (e.g., logic from memory 136), to determine the appropriate stimulation therapy for delivery to the recipient, given the real-time motion of the recipient's head (as presented by the motion signals 160), to replace or supplement the recipient's vestibular function. In this way, the vestibular implant 120 electrically stimulates the nerve cells, bypassing absent or defective vestibular function in a manner that causes the recipient to sensory motion inputs.

The stimulation control signals 162 are provided to a stimulator unit 138. The stimulator unit 138 is component that converts the stimulation control signals 162 into electrical stimulation signals (e.g., current signals) which can then be delivered to the recipient via one or more of the electrode assemblies 148(1)-148(3). The stimulator unit 138 may include, among other elements, one or more current sources.

A problem with certain conventional vestibular implants is that the processing is performed on the basis of only the estimated motion of the recipient with a standard or “catch-all” program/algorithm. However, this type of processing can lead to problems as programs/algorithms used to restore vestibular function while a recipient is walking may not be suitable to restore vestibular function while a recipient is jogging/running. Similarly, programs/algorithms used to restore vestibular function while a recipient is sitting may not be suitable to restore vestibular function while a recipient is driving a car. As such, presented herein are techniques that generate an additional input for use in processing of motion signals to generate the stimulation signals for delivery to a recipient's vestibular system. That is, in certain examples, the stimulation control signals 162 may include one or more adjustments (enhancements) that are based on a specific “activity class” or “activity classification” of the recipient, where the one or more adjustments are incorporated at one or more points within the processing path.

More specifically, as noted above, the vestibular implant 120 comprises the activity classifier 132. As shown in FIG. 3, the activity classifier 132 also receives the one or more motion signals 160 from the one or more motion sensors 130. The activity classifier 132 is configured to analyze the one or more motion signals 160 to generate an “activity class” or “activity classification” for the recipient. As used herein, the “activity class” or “activity classification” is a classification/categorization of the type of activity in which the recipient is currently participating (i.e., the recipient's current activity at the time the one or more motion signals 160 are captured). Stated differently, the activity classifier 132 makes a decision or determination of the recipient's real-time activity.

In FIG. 3, the determined activity class is represented by arrows 166. As shown, the determined activity class 166 can be provided to the processor 134 and/or to the stimulator unit 138. As described further below, the activity class 166 can then be used by the processor 134 and/or to the stimulator unit 138 to adapt/customize the stimulation of the recipient's vestibular for the recipient's current (real-time) activity.

In accordance with certain embodiments presented herein, the activity classifier 132 can classify the recipient's current activity into number of different types of activities. For example, the activity classifier 132 may determine whether the recipient is sleeping, sitting, walking, running, swimming, hiking, bike riding, ascending stairs, descending stairs, etc. However, it is to be appreciate that these specific activity categories are merely illustrative and that, in practice, an activity classifiers could make use of all of these activity classifications, some of these activity classifications, or other activity classifications.

The activity classifier 132 may be implemented in a number of different manners to determine the activity class 166. However, in general, the activity classifier 132 is configured to extract features (i.e., characteristics) from the one or more motion signals. These features may vary depending on the type of analysis being performed (e.g., time or frequency domain analysis) and may include, for example, frequency, measures regarding the static and/or dynamic nature of the signals, etc. The activity classifier 132 operates to determine a category of for the recipient's activity using a type of decision structure (e.g., decision tree, alternative machine learning designs/approaches, and/or other structures that operate based on individual extracted characteristics from the input signals).

In certain embodiments, the activity classifier 132 is configured to analyze the one or more motion signals 160 in the time domain (i.e., analyze the extracted features with respect to time). In other embodiments, the activity classifier 132 is configured to analyze the one or more motion signals 160 in the frequency domain (i.e., analyze the extracted features with respect to frequency, rather than time). In still other embodiments, the activity classifier 132 is configured to analyze the one or more motion signals 160 in the both the frequency and the time domains and correlate the frequency and time domain analysis results to reach a final determination.

In further embodiments, the activity classifier 132 is configured to implement a feature-clustering analysis that utilizes machine learning algorithms (e.g., Hidden Markov models) to determine the recipient's activity class. An example feature-clustering analysis may utilize time domain and/or frequency domain features extracted from the one or more motion signals 160.

In an example time domain analysis, the activity classifier 132 is configured to analyze how the one or more motion signals 160 vary over time. For example, if the signal has a certain low variation (i.e., temporal variation below a predetermined threshold), then the activity classifier 132 may determine that the person is sleeping.

In an example frequency domain analysis, the activity classifier 132 is configured to bandpass filter the one or more motion signals 160 (e.g., using a fast Fourier transform (FFT)) and then analyzes the signal components in the different frequency bands. If, for example, the activity classifier 132 detects most of the activity/energy near a “step frequency” (e.g., 10 Hertz (Hz)), then the activity classifier 132 may determine that person is walking slowly (e.g., activity class is “walking”). If the activity classifier 132 detects most of the activity/energy in a higher frequency band, e.g., 20-30 Hz, then the activity classifier 132 may determine that person is running (e.g., activity class is “running”).

As noted above, regardless of the techniques used, the activity classifier 132 generates/outputs the recipient's real-time activity class 166. Again, as noted above, the determined activity class 166 can be provided to the processor 134 and/or to the stimulator unit 138 and then used to adapt/customize the stimulation of the recipient's vestibular for the recipient's current (real-time) activity.

More specifically, the vestibular implant 120 operates by analyzing the motion signals 160 to determine electrical stimulation signals (e.g., current pulses) that, when delivered to the recipient, restore vestibular function (i.e., help balance the recipient). The processor 134 may be configured to, for example, determine/set the amplitudes/magnitudes of the electrical stimulation signals, determine the stimulation signal timing (i.e., determine current pulse timing), determine the location of the stimulation (e.g., which of the implanted electrodes are used to deliver the stimulation signals), determine the mode of stimulation (e.g., monopolar stimulation, bipolar stimulation, tripolar stimulation, focused multi-polar stimulation, sequential stimulation, etc.), etc.

As noted, certain conventional vestibular implants generate the electrical simulation solely on the basis of the motion signals (i.e., orientation and velocity measures) using a standard program/algorithm. However, this type of processing can lead to problems as programs/algorithms used to restore vestibular function may not be appropriate for all, or even multiple, activities performed by the recipient. For example, it may not be appropriate to simply scale (e.g., increase or decrease) the pulse amplitude, timing, etc., as the orientation and velocity measures change. As such, embodiments presented herein enable the processor 134 to generate the electrical stimulation signals in a manner that is optimized for the recipient's current activity. In particular, the determined activity class 166 can be provided to the processor 134 as an additional input for use in processing of the motion signals 162 to generate the stimulation signals for delivery to a recipient's vestibular system. As such, in accordance with embodiments presented herein, the processor 134 generates the stimulation control signals 162 based not only on the motion signals 160, but also on the determined activity class 166.

The determined activity class 166 functions as contextual data for the operations of the processor 134 and/or to adjust/optimize the operations of stimulator unit 138. For example, through identification of the determined activity class 166 the processor 134 may select the program/algorithms, settings, etc. that are best suited for the recipient's current activity. In one illustrative implementation, the memory 136 stores different processing programs/algorithms, parameters, settings, etc., shown in FIG. 3 as programs 170(1)-170(N), that are each configured for use in generating electrical stimulation signals for different recipient activities. In this example, the determined activity class 166 is used by the processor 134 to select and instantiate, in real-time, the appropriate programs 170(1)-170(N) for using in processing the corresponding received motion signals 160 (i.e., the motion signals used to determine the activity class).

In certain examples, the determined activity class 166 may be used to select/adjust or otherwise set the parameters/attributes of the electrical stimulation pulses (i.e., the stimulation parameters), such as the stimulation rate, stimulation/current pulse width, current or voltage levels, etc. In certain examples, the determined activity class 166 may be used to select/adjust or otherwise set dynamic time domain parameters, such as the automatic gain control parameters (e.g., thresholds, attack time, release time, gain, compression ratio, etc.). In certain examples, the determined activity class 166 may be used to select/adjust or otherwise set dynamic frequency domain parameters, such as filtering parameters (e.g., what frequencies in the sensor data to use for controlling the stimulation parameters). These adjustments may include, for example, selective/dynamic low pass filtering, selective/dynamic high pass filtering, selective/dynamic band pass filtering, etc.

As noted above, in certain embodiments, the determined activity class 166 the processor 134 may select the program/algorithms, settings, etc. that are best suited for the recipient's current activity. In one illustrative example, the determined activity class 166 indicates that the recipient is sleeping. In such an example, the processor 134 and/or the stimulator unit 138 may set the electrical stimulation so as to have a first stimulation rate (e.g., a low pulse rate below a first threshold), apply automatic gain control parameters that result in slow gain changes, and use high pass filtering of the motion sensor signals.

In another illustrative example, the determined activity class 166 indicates that the recipient is walking. In such an example, the processor 134 and/or the stimulator unit 138 may set the electrical stimulation so as to have a second stimulation rate (e.g., a medium pulse rate above the first threshold, but below a second threshold), apply automatic gain control parameters that result in mild gain changes, and use/apply a first band pass filtering of the motion sensor signals (e.g., pass the signals related to walking).

In another illustrative example, the determined activity class 166 indicates that the recipient is running. In such an example, the processor 134 and/or the stimulator unit 138 may set the electrical stimulation so as to have a third stimulation rate (e.g., a high pulse rate above the second threshold), apply automatic gain control parameters that result in fast gain changes, and use/apply a second band pass filtering of the motion sensor signals (e.g., pass the signals related to running).

Embodiments presented herein may use the determined activity class 166 to make a number of different adjustments to the operation of the vestibular implant system. Therefore, it is to be appreciated that the above specific example adjustments made by the processor 134 and/or the stimulator unit 138 based on the determined activity class 166 are merely illustrative.

In summary, FIG. 3 illustrates an example arrangement in which vestibular stimulation can be generated in real-time dependence upon the class, category, or type of activity being performed by a recipient. As a result, the vestibular stimulation is optimized for the recipient's current activity, thereby ensuring proper vestibular inputs to the brain and, accordingly, proper balance for the recipient while performing a wide-range of activities.

FIGS. 2A, 2B, and 3 generally illustrate an arrangement in which the vestibular implant 120 has a totally implanted arrangement, meaning all components of the cochlear vestibular implant 120 are configured to be implanted under skin/tissue 125 of the recipient. Because all components are implantable, vestibular implant 120 operates, for at least a finite period of time, without the need of an external device. As noted, an external device 154 can be used to, for example, charge an internal power source (battery) 139. External device 154 may be a dedicated charger or a multi-function/multi-purpose device. However, it is to be appreciated that the arrangement of FIGS. 2A, 2B, and 3 is illustrative and that vestibular implants in accordance with embodiments may have alternative arrangements in which the functions shown in FIGS. 2A, 2B, and 3 may be split across different devices or components.

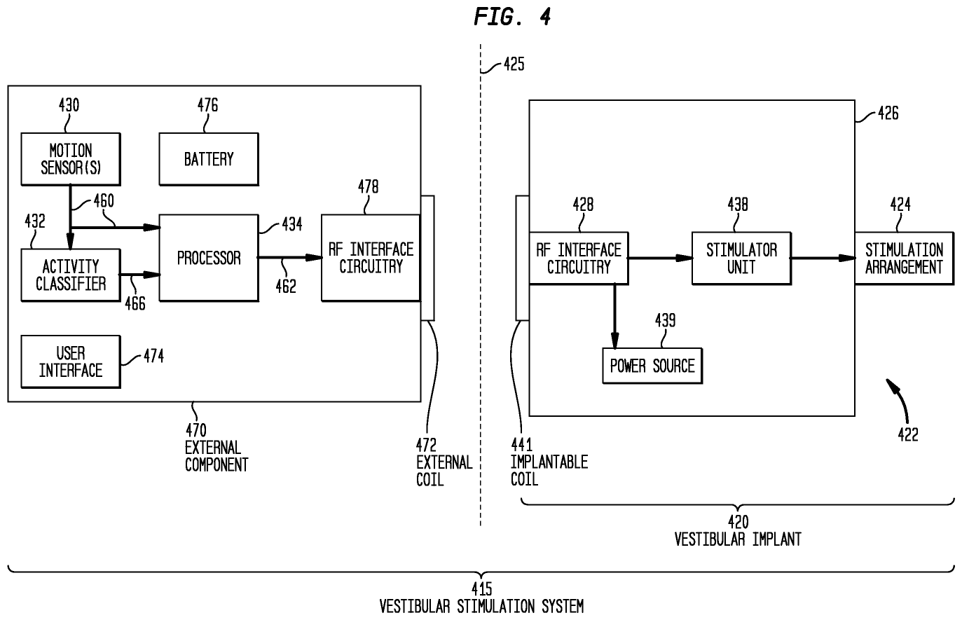

For example, FIG. 4 is block diagram of a vestibular stimulation system 415 comprising an external component 470 and a vestibular implant (implantable component) 420. In this example, the external component 470 is configured to be directly or indirectly attached to the head of the recipient and typically comprises an external coil 472 and, generally, a magnet (not shown in FIG. 4) fixed relative to the external coil 472. The external component 472 also comprises one or more motion sensors 430 (e.g., one or more accelerometers, one or more gyroscopes, etc.) configured generate one or more motion signals 460 from motion of the recipient's head. That is, the one or more motion sensors 430 are similar to sensors 130 of FIGS. 2A, 2B, and 3, which are configured to sense/measure translation and/or rotation of the recipient's head.

The external component 470 also includes, for example, at least one battery 476, a radio-frequency (RF) transceiver 478, an activity classifier 432, a processor 434, and a user interface 474. The activity classifier 432 and the processor 434 may operate similarly to activity classifier 132 and the processor 134 as described above with reference to FIG. 3. In particular, the processor 434 is configured to, based on the one or more motion signals 460 (e.g., data representing recipient's orientation, velocity, etc.), generate stimulation control signals 462 representing electrical stimulation that is to be delivered to the recipient.

The activity classifier 432 also receives the one or more motion signals 460 from the one or more motion sensors 430. The activity classifier 432 is configured to analyze the one or more motion signals 460 and to generate an activity class 466 for the recipient (i.e., determine a classification/categorization of the type of activity in which the recipient is currently participating). Stated differently, the activity classifier 432 makes a decision or determination of the recipient's real-time activity.

In FIG. 4, the determined activity class is represented by arrow 466. As shown, the determined activity class 466 can be provided to the processor 434. Similar to as described above with reference to FIG. 3, the activity class 466 can then be used by the processor 444 to adapt/customize the stimulation of the recipient's vestibular for the recipient's current (real-time) activity. That is, in accordance with embodiments presented herein, the processor 434 generates the stimulation control signals 462 based not only on the motion signals 460, but also on the determined activity class 466.

Each of the activity classifier 432 and the processor 434 may be formed by one or more processors (e.g., one or more Digital Signal Processors (DSPs), one or more uC cores, etc.), firmware, software, etc. arranged to perform operations described herein. That is, the activity classifier 432 and the processor 434 may each be implemented as firmware elements, partially or fully implemented with digital logic gates in one or more application-specific integrated circuits (ASICs), partially in software, etc.

Returning to the example embodiment of FIG. 4, the vestibular implant 420 comprises an implant body (main module) 422 and a vestibular stimulation arrangement 424, both of which are implantable within a recipient (i.e., in implanted under the skin/tissue 425 of a recipient). The implant body 422 generally comprises a hermetically-sealed housing 426 in which Radio-Frequency (RF) interface circuitry 428, a stimulator unit 438, and a rechargeable power source 439. The implant body 422 also includes an internal/implantable coil 441 that is generally external to the housing 426, but which is connected to the RF interface circuitry 428 via a hermetic feedthrough (not shown in FIG. 4).

As noted, the vestibular stimulation arrangement 424 may be similar to vestibular stimulation arrangement 124 described above with reference to FIGS. 2B and 2C. That is, vestibular stimulation arrangement 424 comprises a plurality of electrode assemblies each configured to be inserted into one of the recipient's semicircular canals.

As noted, the external component 470 includes the external coil 472 and the vestibular implant 420 includes implantable coil 441. The coils 472 and 441 are typically wire antenna coils each comprised of multiple turns of electrically insulated single-strand or multi-strand platinum or gold wire. A magnet is fixed relative to each of the external coil 472 and the implantable coil 441, which facilitate the operational alignment of the external coil with the implantable coil. This operational alignment of the coils 472 and 441 enable the external component 470 to transmit data and power to the vestibular implant 420 via a closely-coupled wireless link formed between the coils. In certain examples, the closely-coupled wireless link is a radio frequency (RF) link. However, various other types of energy transfer, such as infrared (IR), electromagnetic, capacitive and inductive transfer, may be used to transfer the power and/or data from an external component to an implantable component and, as such, FIG. 1B illustrates only one example arrangement.

As noted above, the processor 434 generates the stimulation control signals 462 based on the motion signals 460 and the determined activity class 466. In the embodiment of FIG. 4, the stimulation control signals 462 are provided to the RF interface circuitry 478, which transcutaneously transfers the stimulation control signals 462 (e.g., in an encoded manner) to the vestibular implant 420 via external coil 472 and implantable coil 441. That is, the stimulation control signals 462 are received at the RF interface circuitry 428 via implantable coil 441 and provided to the stimulator unit 438. The stimulator unit 438 is configured to utilize the stimulation control signals 462 to generate electrical stimulation signals (e.g., current signals) for delivery to the recipient's vestibular system via the stimulation arrangement 424.

As noted, FIG. 4 illustrates an arrangement in which the vestibular stimulation system 415 comprises a vestibular implant 420 and an external component 470 that provides both power and stimulation control signals to the vestibular implant 420. It is to be appreciated that embodiments of the present invention may be implemented with vestibular implant having alternative arrangements.

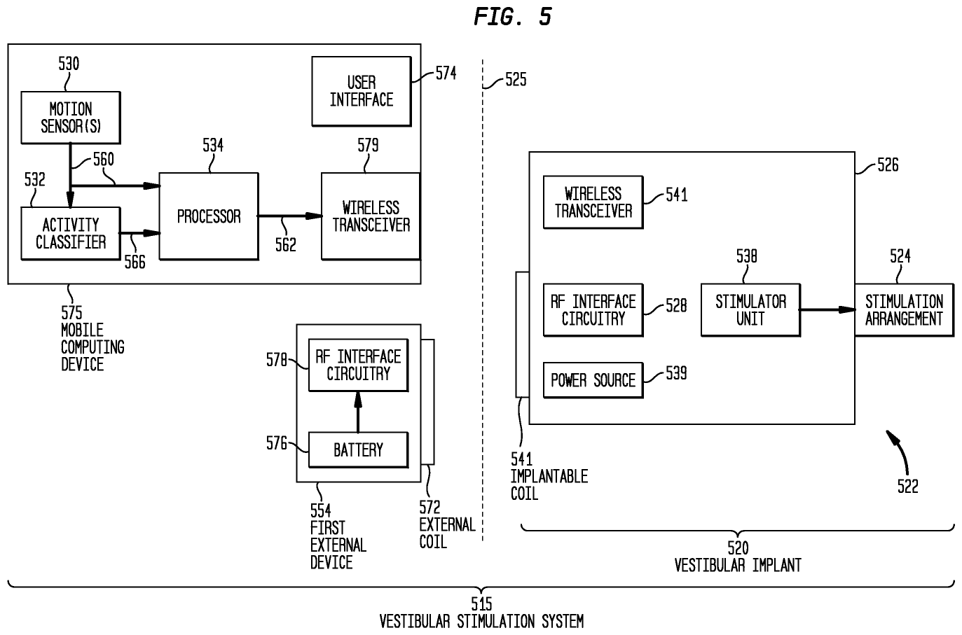

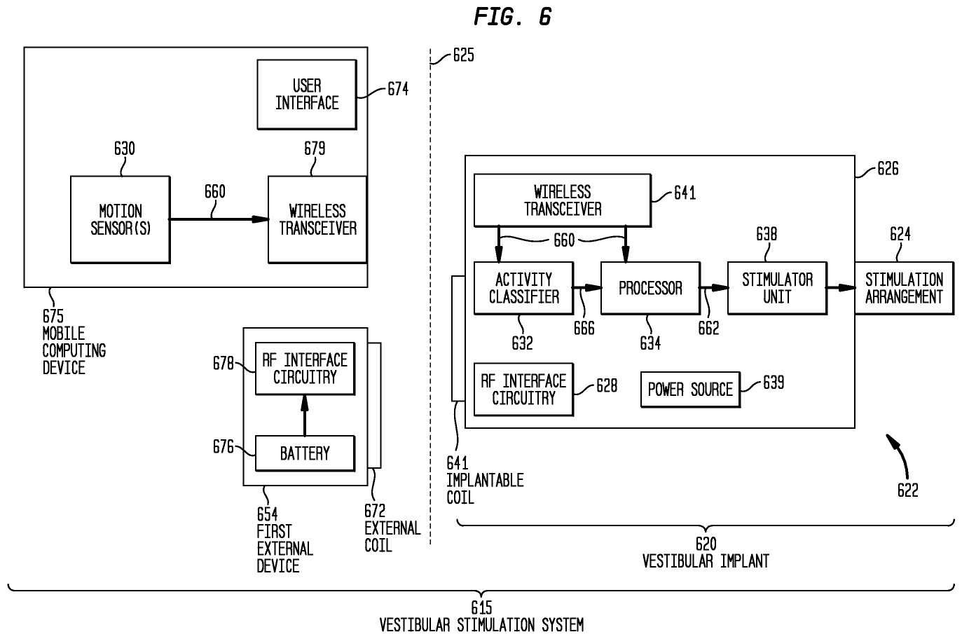

For example, FIG. 5 illustrates another vestibular stimulation system 515 that comprises a vestibular implant 520, a first external device 554, and a second external device 575. In this example, the second external device 575 is a mobile computing device, such as mobile phone, a wearable device (e.g., smartwatch, fitness tracker device, etc.) configured to be worn by, or carried by, a recipient.

As shown, the first external device 554 comprises an external coil 572 and, generally, a magnet (not shown in FIG. 5) fixed relative to the external coil 572. The first external device 554 also includes, for example, at least one battery 576, a radio-frequency (RF) transceiver 578.

The mobile computing device 575 may comprise a number of functional elements to perform a number of different functions/operations. For ease of illustration, FIG. 5 only illustrates components of mobile computing device 575 related to the techniques presented herein. In particular, FIG. 5 illustrates that the mobile computing device 575 comprises one or more motion sensors 530 (e.g., one or more accelerometers, one or more gyroscopes, etc.) configured generate one or more motion signals 560 from motion of the recipient. The one or more motion sensors 530 may be similar to sensors 130 of FIGS. 2A, 2B, and 3, and are configured to sense/measure translation and/or rotation of the recipient's body and/or one or more of recipient's body parts.

The mobile computing device 575 also includes, for example, an activity classifier 532, a processor 534, a user interface 574, and a wireless transceiver 579. The activity classifier 532 and the processor 534 may operate similarly to activity classifier 132 and the processor 134 as described above with reference to FIG. 3. In particular, the processor 534 is configured to, based on the one or more motion signals 560 (e.g., data representing recipient's orientation, velocity, etc.), generate stimulation control signals 562 representing electrical stimulation that is to be delivered to the recipient.

The activity classifier 532 also receives the one or more motion signals 560 from the one or more motion sensors 530. The activity classifier 532 is configured to analyze the one or more motion signals 560 and to generate an activity class 566 for the recipient (i.e., determine a classification/categorization of the type of activity in which the recipient is currently participating). Stated differently, the activity classifier 532 makes a decision or determination of the recipient's real-time activity.

In FIG. 5, the determined activity class is represented by arrow 566. As shown, the determined activity class 566 can be provided to the processor 534. Similar to as described above with reference to FIG. 3, the activity class 566 can then be used by the processor 544 to adapt/customize the stimulation of the recipient's vestibular for the recipient's current (real-time) activity. That is, in accordance with embodiments presented herein, the processor 534 generates the stimulation control signals 562 based not only on the motion signals 560, but also on the determined activity class 566.

Each of the activity classifier 532 and the processor 534 may be formed by one or more processors (e.g., one or more Digital Signal Processors (DSPs), one or more uC cores, etc.), firmware, software, etc. arranged to perform operations described herein. That is, the activity classifier 532 and the processor 534 may each be implemented as firmware elements, partially or fully implemented with digital logic gates in one or more application-specific integrated circuits (ASICs), partially in software, etc.

Returning to the example embodiment of FIG. 5, the vestibular implant 520 comprises an implant body (main module) 522 and a vestibular stimulation arrangement 524, both of which are implantable within a recipient (i.e., in implanted under the skin/tissue 525 of a recipient). The implant body 522 generally comprises a hermetically-sealed housing 526 in which Radio-Frequency (RF) interface circuitry 528, a stimulator unit 538, a rechargeable power source 539, and a wireless transceiver 541 are disposed. The implant body 522 also includes an internal/implantable coil 541 that is generally external to the housing 526, but which is connected to the RF interface circuitry 528 via a hermetic feedthrough (not shown in FIG. 5).

The vestibular stimulation arrangement 524 may be similar to vestibular stimulation arrangement 124 described above with reference to FIGS. 2B and 2C. That is, vestibular stimulation arrangement 524 comprises a plurality of electrode assemblies each configured to be inserted into one of the recipient's semicircular canals.

As noted, the first external device 554 includes the external coil 572 and the vestibular implant 520 includes implantable coil 541. The coils 572 and 541 are typically wire antenna coils each comprised of multiple turns of electrically insulated single-strand or multi-strand platinum or gold wire. In certain examples, a magnet is fixed relative to each of the external coil 572 and the implantable coil 541, which facilitate the operational alignment of the external coil with the implantable coil. This operational alignment of the coils 572 and 541 enable the external component 570 to transmit power to the vestibular implant 520 via a closely-coupled wireless link formed between the coils (e.g., an RF link). That is, in this example, the first external device 554 is a charging device for recharging the implantable power source 539. The first external device 554 may be used, for example, while the recipient is sleeping to recharge the implant power source 539.

As noted above, in the embodiment of FIG. 5, the processor 534 in the mobile computing device 575 generates the stimulation control signals 562 based on the motion signals 560 and the determined activity class 566. In the embodiment of FIG. 5, the stimulation control signals 562 are provided to the wireless transceiver 579, which wirelessly sends the stimulation control signals 562 (e.g., in an encoded manner) to the vestibular implant 520 via wireless transceiver 541. That is, the stimulation control signals 562 are received at the wireless transceiver 541 and provided to the stimulator unit 538. The stimulator unit 538 is configured to utilize the stimulation control signals 562 to generate electrical stimulation signals (e.g., current signals) for delivery to the recipient's vestibular system via the stimulation arrangement 524.

As noted, FIG. 5 illustrates an arrangement in which the motion sensing, processing, and activity classification are all performed by an external mobile computing device. It is to be appreciated that, in other embodiments, these functions may be split between an external device and the vestibular implant.

For example, FIG. 6 another vestibular stimulation system 615 that comprises a vestibular implant 620, a first external device 654, and a second external device 675. In this example, the second external device 675 is a mobile computing device, such as mobile phone, a wearable device (e.g., smartwatch, fitness tracker device, etc.) configured to be worn by, or carried by, a recipient.

The first external device 654 comprises an external coil 672 and, generally, a magnet (not shown in FIG. 6) fixed relative to the external coil 672. The first external device 654 also includes, for example, at least one battery 676, a radio-frequency (RF) transceiver 678.

The mobile computing device 675 may comprise a number of functional elements to perform a number of different functions/operations. For ease of illustration, FIG. 6 only illustrates components of mobile computing device 675 related to the techniques presented herein. In particular, FIG. 6 illustrates that the mobile computing device 675 comprises one or more motion sensors 630 (e.g., one or more accelerometers, one or more gyroscopes, etc.) configured generate one or more motion signals 660 from motion of the recipient. The one or more motion sensors 630 may be similar to sensors 130 of FIGS. 2A, 2B, and 3, and are configured to sense/measure translation and/or rotation of the recipient's body and/or one or more of recipient's body parts. The mobile computing device 675 also includes a wireless transceiver 679 and a user interface 674.

The vestibular implant 620 comprises an implant body (main module) 622 and a vestibular stimulation arrangement 624, both of which are implantable within a recipient (i.e., in implanted under the skin/tissue 625 of a recipient). The implant body 622 generally comprises a hermetically-sealed housing 626 in which Radio-Frequency (RF) interface circuitry 628, an activity classifier 632, a processor 634, a stimulator unit 638, a rechargeable power source 639, and a wireless transceiver 641 are disposed. The implant body 622 also includes an internal/implantable coil 641 that is generally external to the housing 626, but which is connected to the RF interface circuitry 628 via a hermetic feedthrough (not shown in FIG. 6).

The vestibular stimulation arrangement 624 may be similar to vestibular stimulation arrangement 124 described above with reference to FIGS. 2B and 2C. That is, vestibular stimulation arrangement 624 comprises a plurality of electrode assemblies each configured to be inserted into one of the recipient's semicircular canals.

As noted, the first external device 654 includes the external coil 672 and the vestibular implant 620 includes implantable coil 641. The coils 672 and 641 are typically wire antenna coils each comprised of multiple turns of electrically insulated single-strand or multi-strand platinum or gold wire. In certain examples, a magnet is fixed relative to each of the external coil 672 and the implantable coil 641, which facilitate the operational alignment of the external coil with the implantable coil. This operational alignment of the coils 672 and 641 enable the external component 670 to transmit power to the vestibular implant 620 via a closely-coupled wireless link formed between the coils (e.g., an RF link). That is, in this example, the first external device 654 is a charging device for recharging the implantable power source 639. The first external device 654 may be used, for example, while the recipient is sleeping to recharge the implantable power source 639.

As noted above, in the embodiment of FIG. 6, the mobile computing device 675 includes one or more motion sensors 630 configured to generate motion signals 660. In the embodiment of FIG. 6, the one or more motion signals 660 are provided to the wireless transceiver 679, which wirelessly sends the one or more motion signals 660 (e.g., in an encoded manner) to the vestibular implant 620 via wireless transceiver 641. That is, the one or more motion signals 660 are received at the wireless transceiver 641 and provided to the activity classifier 632 and the processor 634.

The activity classifier 632 and the processor 634 may operate similarly to activity classifier 132 and the processor 134 as described above with reference to FIG. 3. In particular, the processor 634 is configured to, based on the one or more motion signals 660 (e.g., data representing recipient's orientation, velocity, etc.), generate stimulation control signals 662 representing electrical stimulation that is to be delivered to the recipient.

The activity classifier 632 also receives the one or more motion signals 660 from the one or more motion sensors 630. The activity classifier 632 is configured to analyze the one or more motion signals 660 and to generate an activity class 666 for the recipient (i.e., determine a classification/categorization of the type of activity in which the recipient is currently participating). Stated differently, the activity classifier 632 makes a decision or determination of the recipient's real-time activity.

In FIG. 6, the determined activity class is represented by arrow 666. As shown, the determined activity class 666 can be provided to the processor 634. Similar to as described above with reference to FIG. 3, the activity class 666 can then be used by the processor 644 to adapt/customize the stimulation of the recipient's vestibular for the recipient's current (real-time) activity. That is, in accordance with embodiments presented herein, the processor 634 generates the stimulation control signals 662 based not only on the motion signals 660, but also on the determined activity class 666.

The stimulation control signals 662 are provided to the stimulator unit 638. The stimulator unit 638 is configured to utilize the stimulation control signals 662 to generate electrical stimulation signals (e.g., current signals) for delivery to the recipient's vestibular system via the stimulation arrangement 624.

Each of the activity classifier 632 and the processor 634 may be formed by one or more processors (e.g., one or more Digital Signal Processors (DSPs), one or more uC cores, etc.), firmware, software, etc. arranged to perform operations described herein. That is, the activity classifier 632 and the processor 634 may each be implemented as firmware elements, partially or fully implemented with digital logic gates in one or more application-specific integrated circuits (ASICs), partially in software, etc.

It is to be appreciated that the embodiments of FIGS. 2A, 2B, 3, 4, 5, and 6 are illustrative of arrangements for vestibular implants and vestibular stimulation systems in accordance with embodiments presented herein. It is also to be appreciated that the embodiments of FIGS. 2A, 2B, 3, 4, 5, and 6 are not mutually exclusive and that other arrangements are possible. For example, the vestibular implant of FIGS. 2A, 2B, and 3 could also operate with a mobile computing device, such as the mobile computing devices 575 and 675 of FIGS. 5 and 6, respectively. In such embodiments, the activity classifier 132 could generate the activity classification 166 based on motion signals generated by the motion sensors 130 and/or based on motion signals generated by the motion sensors 530 or 630. Similarly, in such embodiments, the processor 134 could generate the stimulation control signals 662 based on motion signals generated by the motion sensors 130 and/or based on motion signals generated by the motion sensors 530 or 630, as well as the activity classification 166.

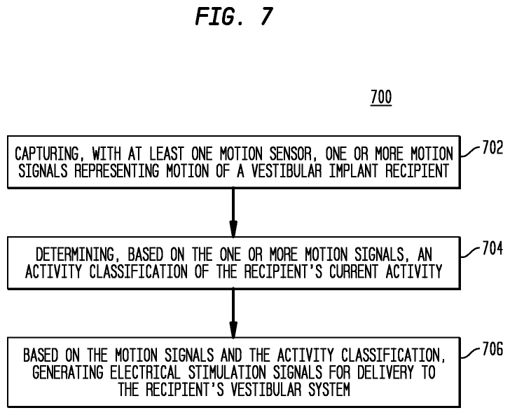

FIG. 7 is a flowchart of a method 700 in accordance with embodiments presented herein. Method 700 begins at 702 where at least one motion sensor captures one or more motion signals representing motion of a vestibular implant recipient. At 704, the one or more motion signals are used to determine an activity classification of the recipient's current activity. At 706, the motion signals and the activity classification are used to generate electrical stimulation signals for delivery to the recipient's vestibular system.

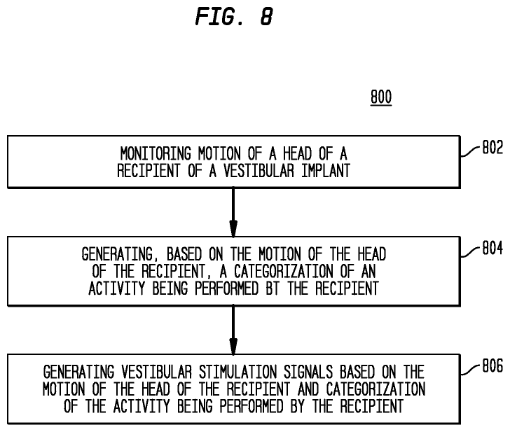

FIG. 8 is a flowchart of a method 800 in accordance with embodiments presented herein. Method 800 begins at 802 which monitoring of the motion of a head of a recipient of a vestibular implant. At 804, the motion of the head of the recipient is used to generate a categorization of an activity being performed by the recipient. At 806, vestibular stimulation signals are generated based on the motion of the head of the recipient and categorization of the activity being performed by the recipient.

It is to be appreciated that the above described embodiments are not mutually exclusive and that the various embodiments can be combined in various manners and arrangements.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

1. A method, comprising:
capturing, with at least one motion sensor, one or more motion signals representing motion of a vestibular implant recipient;
determining, based on the one or more motion signals, an activity classification of the recipient's current activity; and
based on the one or more motion signals and the activity classification, generating electrical stimulation signals for delivery to a vestibular system of the recipient.

2. The method of claim 1, further comprising:
delivering the electrical stimulation signals to the vestibular system via one or more electrodes implanted in one or more semi-circular canals of the vestibular system.

3. The method of claim 1, wherein capturing one or more motion signals representing motion of a vestibular implant recipient comprises:
capturing the one or more motion signals with at least one accelerometer.

4. The method of claim 1, wherein capturing one or more motion signals representing motion of a vestibular implant recipient comprises:
capturing the one or more motion signals with at least one gyroscope.

5. The method of claim 1, wherein capturing one or more motion signals representing motion of a vestibular implant recipient comprises:
capturing the one or more motion signals with at least one motion sensor implanted in the head of the recipient.

6. The method of claim 1, wherein capturing one or more motion signals representing motion of a vestibular implant recipient comprises:
capturing the one or more motion signals with at least one motion sensor external to the body of the recipient.

7. The method of claim 1, wherein determining the activity classification of the recipient's current activity comprises:
analyzing the one or more motion signals in the time domain to generate the activity classification.

8. The method of claim 1, wherein determining the activity classification of the recipient's current activity comprises:
analyzing the one or more motion signals in the frequency domain to generate the activity classification.

9. The method of claim 1, wherein determining the activity classification of the recipient's current activity comprises:
analyzing the one or more motion signals in the time domain;
analyzing the one or more motion signals in the frequency domain; and
correlating the time domain analysis and the frequency domain analysis of the one or more motion signals to generate the activity classification.

10. The method of claim 1, wherein determining the activity classification of the recipient's current activity comprises:
performing a feature-clustering analysis that utilizes one or more machine learning algorithms to generate the activity classification.

11. The method of claim 1, wherein generating electrical stimulation signals for delivery to the vestibular system based on the one or more motion signals and the activity classification, comprises:
setting one or more of a stimulation pulse rate, a stimulation pulse width, a current level, or a voltage of the electrical stimulation signals based on the activity classification.

12. The method of claim 1, wherein generating electrical stimulation signals for delivery to the vestibular system based on the one or more motion signals and the activity classification, comprises:
applying automatic gain control to the one or more motion signals; and
setting one or more parameters of the automatic gain control based on the activity classification.

13. The method of claim 1, wherein generating electrical stimulation signals for delivery to the vestibular system based on the one or more motion signals and the activity classification, comprises:
applying one or more filtering operations to the one or more motion signals; and
setting one or more parameters of the filtering operations based on the activity classification.

14. A vestibular stimulation system, comprising:
one or more motion sensors configured to convert motion of a recipient of the vestibular stimulation system into one or more motion signals;
at least one activity classifier configured to generate, based on the one or more motion signals, an activity classification representing a real-time activity of the recipient;
at least one processor configured to generate stimulation control signals based on the motion signals and the activity classification; and
a stimulator unit configured to convert the stimulation control signals into electrical stimulation signals for delivery to the recipient's vestibular system.

15. The vestibular stimulation system of claim 14, further comprising:
one or more electrode assemblies configured to be implanted in one or more semicircular canals of the recipient, wherein each of the one or more electrode assemblies comprises a plurality of electrodes disposed in a carrier member.

16. The vestibular stimulation system of claim 14, wherein the one or more motion sensors comprise at least one accelerometer.

17. The vestibular stimulation system of claim 14, wherein the one or more motion sensors comprise at least one gyroscope.

18. The vestibular stimulation system of claim 14, wherein at least one of the one or more motion sensors is configured to be implanted in the head of the recipient.

19. The vestibular stimulation system of claim 14, wherein at least one of the one or more motion sensors is external to the body of the recipient.

20. The vestibular stimulation system of claim 14, wherein the vestibular stimulation system comprises an implantable component and an external device, and wherein the activity classifier, the at least one processor, and the stimulator unit are disposed in the implantable component.

21. The vestibular stimulation system of claim 14, wherein the vestibular stimulation system comprises an implantable component and an external device, and wherein least one of the one or more motion sensors, the activity classifier, and the at least one processor are disposed in the external component.

22. The vestibular stimulation system of claim 14, wherein the activity classifier is configured to:
extract a plurality of features from the one or more motion signals; and
analyze the plurality of extracted features with respect to time to generate the activity classification.

23. The vestibular stimulation system of claim 14, wherein the activity classifier is configured to:
extract a plurality of features from the one or more motion signals; and
analyze the plurality of extracted features with respect to frequency to generate the activity classification.

24. The vestibular stimulation system of claim 14, wherein the activity classifier is configured to:
extract a plurality of features from the one or more motion signals;
analyze the plurality of extracted features with respect to time;
analyze the plurality of extracted features with respect to frequency; and
correlating results of analyzing of the plurality of extracted features with respect to time with the analysis of the plurality of extracted features with respect to frequency to generate the activity classification.

25. The vestibular stimulation system of claim 14, wherein the activity classifier is configured to:
extract a plurality of features from the one or more motion signals; and
perform a feature-clustering analysis on the plurality of features using one or more machine learning algorithms to generate the activity classification.

26. The vestibular stimulation system of claim 14, wherein the activity classifier is configured to:
extract a plurality of features from the one or more motion signals; and
analyze the plurality of features with a decision tree structure to generate the activity classification.

27. The vestibular stimulation system of claim 14, wherein the at least one processor is configured to use the activity classification to select, from a plurality of programs, a first program for use in processing the one or more motion signals to generate the stimulation control signals.

28. The vestibular stimulation system of claim 14, wherein to generate stimulation control signals based on the one or more motion signals and the activity classification, the at least one processor is configured to:
set one or more of a stimulation pulse rate, a stimulation pulse width, a current level, or a voltage of the electrical stimulation signals based on the activity classification.

29. The vestibular stimulation system of claim 14, wherein to generate stimulation control signals based on the one or more motion signals and the activity classification, the at least one processor is configured to:
apply automatic gain control to the one or more motion signals; and
set one or more parameters of the automatic gain control based on the activity classification.

30. The vestibular stimulation system of claim 14, wherein to generate stimulation control signals based on the one or more motion signals and the activity classification, the at least one processor is configured to:
apply one or more filtering operations to the one or more motion signals; and
set one or more parameters of the filtering operations based on the activity classification.

31. A method, comprising:
monitoring motion of a head of a recipient of a vestibular implant;
generating, based on the motion of the head of the recipient, a categorization of an activity being performed by the recipient; and
generating vestibular stimulation signals based on the motion of the head of the recipient and categorization of the activity being performed by the recipient.

32. The method of claim 31, further comprising:
delivering the vestibular stimulation signals to a vestibular system of the recipient via one or more electrodes implanted in one or more semi-circular canals of the vestibular system.

33. The method of claim 31, wherein monitoring the motion of the head of the recipient of the vestibular implant comprises:
monitoring the motion of the head of the recipient of the vestibular implant with at least one motion sensor implanted in the head of the recipient.

34. The method of claim 31, wherein monitoring the motion of the head of the recipient of the vestibular implant comprises:
monitoring the motion of the head of the recipient of the vestibular implant with at least one motion sensor external to the head of the recipient.
